(19) United States
(12) Patent Application Publication
(10) Pub. No.: US 2020/0330627 A1
(43) Pub. Date: Oct. 22, 2020

(54) APPARATUS FOR STERILISING ROOT CANAL FILLING MATERIAL WITH UV LIGHT

(71) Applicants: Shanon Patel, London (GB); Andrew Dawood, London (GB)

(72) Inventors: Shanon Patel, London (GB); Andrew Dawood, London (GB)

(21) Appl. No.: 16/641,033

(22) PCT Filed: Aug. 20, 2018
(86) PCT No.: PCT/GB2018/052349

§ 371 Date: Feb. 21, 2020

(30) Foreign Application Priority Data
Aug. 21, 2017 (GB) 1713352.1
Mar. 20, 2018 (GB) 1804463.6

(51) Int. Cl. A61L 2/00; A61L 2/24; A61C 5/50

(52) U.S. Cl. CPC A61L 2/0047; A61L 2/24; A61C 5/50; A61L 2202/21; A61L 2202/11; A61L 2202/121; A61L 2202/14; A61L 2202/122

(57) ABSTRACT

Apparatus comprising: a container; means for emitting UV light (20) located in the container; at least one receptacle (26a) for location in the container and for retaining root canal filling material comprising a plurality of cones (30), such that each cone is disposed so as not to contact other of the cones, wherein the emitting means and the at least one receptacle are configured such that UV light emitted by the emitting means is incident on retained cones with anti-bacterial effect. Cooling means may be provided between a base (11) of the container and the support member (12).

### FIELD OF THE INVENTION

The invention relates to apparatus including means for emitting UV light on root canal filling material comprising cones, particularly thermoplastic cones, with antibacterial effect.

### BACKGROUND

Root canal treatment (endodontics) is a branch of dentistry in which a root canal system of a tooth is cleaned in order to remove dental pulp and tissue, which may be inflamed, necrotic, and/or infected. During treatment, the root canal system is prepared, debrided and disinfected by means of various instruments and disinfection agents to result in a prepared canal into which an inert root canal filling material may be obturated to prevent the disinfected root canal system from being re-infected.

The root canal filling material may be in the form of a cone coated in sealer. The cone is typically formed of thermoplastic and may be referred to as a “gutta percha point”. The sealer may be applied using the cone to the canal. A heated instrument is used to remove excess cone. The cone is inserted and is malleable such that it can be packed down with a plugger.

A cone should be sterile before it is put into the root canal, but the cone is often contaminated by microorganisms. In addition, freshly unpacked materials, for example mixing pads, rubber dams and damming materials, which are commonly used in disinfection and subsequent obturation of the disinfected root canal space have also been shown to be contaminated with microorganisms. The microbial count on these endodontic materials increases with time. Bacterial species identified on these materials typically includes bacteria implicated in the failure of root canal treatment. It has been suggested that nosocomial infection from the dental surgery environment from endodontic materials introduced into the root canal may be cause/contribute to this root canal failure.

Given that the entire root canal treatment process is intended to eliminate bacteria and seal the root canal, the introduction of a contaminated cone into the cleaned and prepared root canal is clearly inappropriate. In addition, the presence of microorganisms on associated endodontic materials may result in unintentional contamination of the root canal system.

Various chemical agents are used for the decontamination of root canals or root canal instrumentation; however, these may not be suitable for use with cones and/or associated endodontic materials.

Use of UV light to kill microorganisms in hospitals using large immovable cabinets is known. Known documents, CN204814948U, CN2037242 and EP2953655, disclose boxes including UV lamps, but none is suitable for retaining gutta percha points. It is an object of the present invention to provide apparatus for interoperative use that can be used to sterilise gutta percha points and thus reduce likelihood of infection.

### SUMMARY OF THE INVENTION

According to an aspect of the present invention, there is provided apparatus comprising: a container; means for emitting UV light located in the container; at least one receptacle for location in the container and for retaining root canal filling material comprising a plurality of cones, wherein the emitting means and the at least one receptacle are configured such that UV light emitted by the emitting means is incident on retained cones with anti-bacterial effect.

Thus, the root canal filling material may be sterile when taken from the container, reducing risk of infection. The cones are not in contact with each other, that is, they are spaced so that light can be incident all around each cone.

The apparatus may further comprise a member transparent to UV light emitted from the emitting means extending across an interior of the container, wherein, in use of the container, the emitting means is located beneath the member and the at least one receptacle is located on top of the member. The at least one receptacle may be located to rest on an upper side of the member, such that the member supports the at least one receptacle.

The member and parts of the at least one side wall that are, in use, above the member, may provide a space in which the at least one receptacle and any root canal filling material located in the at least one receptacle is located. The space forms an armamentarium. The member preferably extends across the interior of the container to seal the armamentarium with respect to a side of the member on which the emitting means is located.

Further optional and/or preferred features of the apparatus are defined in the dependent claims.

According to a further aspect of the present invention, there is provided apparatus comprising: a container; means for emitting UV light located in the container; a member transparent to UV light emitted from the emitting means extending across an interior of the container, wherein, in use, the emitting means is located beneath the member, wherein the member extends across the interior of the container such that passage of bacteria from beneath the member to above the member is prevented; cooling means located, in use of the apparatus, beneath the member and arranged to dissipate heat generated by the emitting means.

The apparatus may be used in surgery to store and sterilise any articles used in surgery. The apparatus is particularly useful where the articles are sensitive to temperature, since the apparatus can be configured to keep the temperature in a space above the member cool, or below a predetermined threshold.

The apparatus may further comprise a closure to close the container, so as to prevent ingress of bacteria into the space.

The apparatus may further comprise at least one receptacle for location in the container above the member and for retaining one or more articles or materials, wherein the emitting means and the at least one receptacle are configured such that UV light emitted by the emitting means is incident on retained articles with anti-bacterial effect. The at least one receptacle may be removable from the container. The at least one receptacle may be transparent to UV light emitted by the emitting means. There may be a plurality of receptacles. Although such articles and materials could be located directly on the transparent member, use of the at least one receptacles prevents build up of dirt on the transparent member.

The member and parts of at least one side wall that are, in use, above the member, provide a space in which the at least one receptacle and any article located in the at least one receptacle are located.

The container has a base and the emitting means may be located between the member and the base. When the base of the container is located on a horizontal surface, the member is preferably horizontal.

The apparatus may further comprise means for supporting the member, for example one or more projections extending inwardly from the at least one side wall.

The apparatus may further comprise at least one air hole in the at least one side permitting air flow between outside of the container and the cooling means. The apparatus may further comprise control means operatively coupled to the emitting means, and configured to control starting and stopping of emitting of UV light by the emitting means.

The apparatus may further comprise, operatively coupled to the control means, means for detecting opening of the container, wherein the control means is configured to control the emitting means so as to stop emitting UV light in response to detecting opening of the container. The means for detecting may also be configured to detect locating of the closure to close the container, wherein the control means is configured to control the emitting means so as to start emitting UV light in response to detecting closing of the container.

The control means may be configured with a timer, wherein, when the control means determines to control the emitting means to emit UV light, the control means also starts the timer, and wherein the control means is configured to control the emitting means so as to stop emitting UV light when a predetermined time period has ended.

The control means may be configured to start the timer each time the control means controls the emitting means so as to start emitting UV light. The control means may be operatively coupled to the cooling means, and be configured to cause starting and stopping of the cooling means with starting and stopping of emitting of UV light by the emitting means.

The apparatus may further comprise a thermostat operatively coupled to the control means and located to determine information indicative of the temperature of the at least one receptacle. In this case, the control means may be configured to cause starting and stopping of the cooling means dependent on the information received from the thermostat.

### BRIEF DESCRIPTION OF FIGURES

For better understanding of the present invention, embodiments of the present invention will now be described, by way of example only, in which:

FIG. 1 is a perspective view of a sterilising unit in accordance with embodiments of the invention, with a lid partially open;

FIG. 2 is a cross-sectional, perspective view of the unit, with removable modules, hinges and a lid absent;

FIG. 3 is the cross-sectional, perspective view of the unit, with three modules located in the unit; and

FIG. 4 is a view of another module for location in the sterilising unit; and

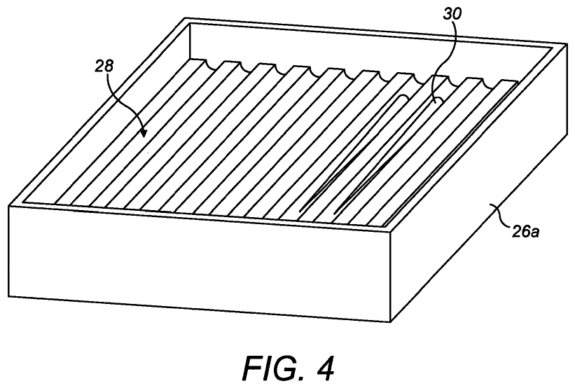

FIG. 5 shows diagrammatically elements of the sterilising unit that are electrically coupled.

### DETAILED DESCRIPTION OF EMBODIMENTS

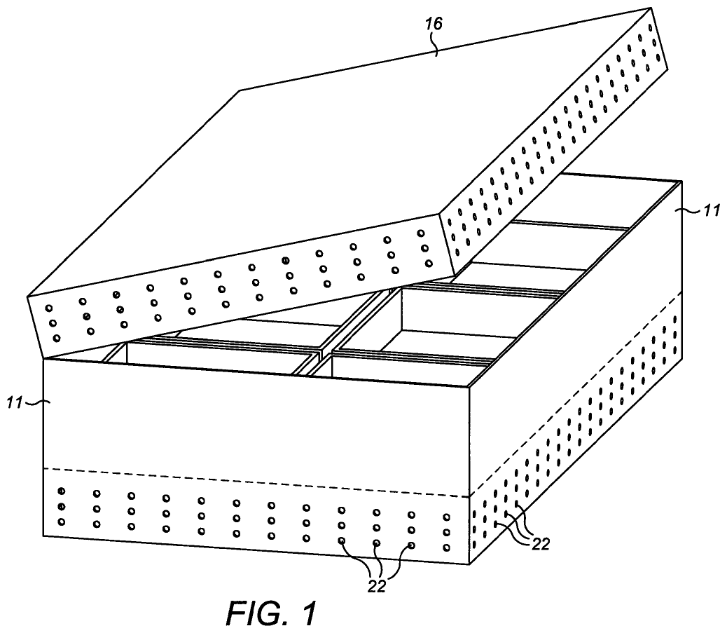

Referring to FIG. 1, in an embodiment a sterilising unit comprising a container 10 and a transparent plate 12, a cooling unit 14 and a UV light source all located in the container. The sterilising unit also comprises at least one receptacle 26a-d for retaining root canal filling material comprising a plurality of cones. The sterilising unit has a particular orientation for use and terms such as “upper” and “beneath” are to be construed accordingly.

The container 10 has a base 11 and four sides 13 extending from the base 11 in a rectangular manner. The base 11 is flat enabling the sterilising unit to be stably located on a horizontal surface, so that, when so located, the sides 13 extend upwardly. The sides 13 are opaque with respect to UV light. In variant embodiments, the container need not be rectangular; for example, the container may be circular, in which case it only has one side.

The transparent plate 12 extends across an interior of the container 10 to support the at least one receptacle 26a-d. The transparent plate 12 is transparent to UV light emitted from the UV light source. The transparent plate 12 and first parts of the sides 13 above the transparent plate 12 define a space that serves as an armamentarium in which the receptacles 26a-d are located. The transparent plate 12 is formed of fused quartz silica, although other materials may be used in alternative embodiments.

The container 10 is configured to support the transparent plate 12. To this end, ridges 18 extend from the sides 13 each at a same distance from the base 11. The ridges 18 are located to support the transparent plate 12 such that the transparent plate 12 is horizontal when the base 11 of the sterilising unit is located on a horizontal surface. In variant embodiments, the transparent plate 12 may be otherwise retained in the container 10. For example, instead of the ridges 18, the sides 13 of the container 10 may be provided with grooves in which edges of the transparent plate 12 are located.

Preferably, the container 10 and the transparent plate 12 are respectively configured to prevent or impede passage of bacteria from beneath the transparent plate 12 to the armamentarium. The ends of the transparent plate 12 may be rubberised and the sides 13 and the transparent plate 12 respectively shaped to form a seal. Additionally or alternatively, the ridges 18 may be rubberised.

The sterilising unit also includes a closure in the form of a lid 16 hingedly attached to one of the sides 13 at hinge 17. The lid 16 and the container 10 are respectively configured such the lid 16, when closed, seals the armamentarium with respect to ambient air. In variant embodiments, the sterilising unit may include other kinds of closure mechanisms in place of the hinged lid 16.

The UV light source is configured to emit UV light, particularly UVC light at 240 to 280 nanometres, that has sterilising, or at least antibacterial, effect on articles located in the armamentarium. The UV light source is preferably arranged so that articles stored in the armamentarium have all round exposure to the UV light. The UV light has sterilising effect as it breaks down proteins including microbial DNA exposed to the UV light. Also, UV light causes oxygen to ozonate and the resultant ozone may have sterilising effect on microorganisms. The UV light source is located on a lower side of the transparent plate 12 and is configured so that light emitted by the UV light source is directed upwards through the transparent plate 12. The UV light source is in the form of a plurality of LEDs 20. Other sources of UV light may be used, but LEDs generate little heat in view of their light output.

First parts of the side walls 13 that define the armamentarium may have a reflective coating on them to prevent absorption of UV light by the walls 13 and for better exposure of articles in the armamentarium to the UV light.

The cooling unit 14 is configured to absorb heat generated by the LEDs 20, such that the temperature in the armamentarium does not significantly rise or remains below a predetermined temperature. The cooling unit 14 is located between the LEDs 20 and the base 11 and comprises a heat sink and fan arrangement.

Second parts of the sides 13 of the container 10 that are beneath the transparent plate 12 have air holes 22 through them to allow ventilation to the heat sink. A fan is provided to cause passage of air over the cooling unit 14. Preferably, the base 11 and/or the side walls 13 are formed of a heat conductive material, for example a metal, to aid in dissipation of heat generated by the LEDs 20.

Although not essential and not indicated in the Figures, the lid may have a thin heat-conducting liner and be hollow. Holes in the lid enable flow of air from outside of the sterilising unit to the hollow interior of the lid. Heat in the armamentarium can thus be transmitted to the exterior of the sterilising unit via the liner.

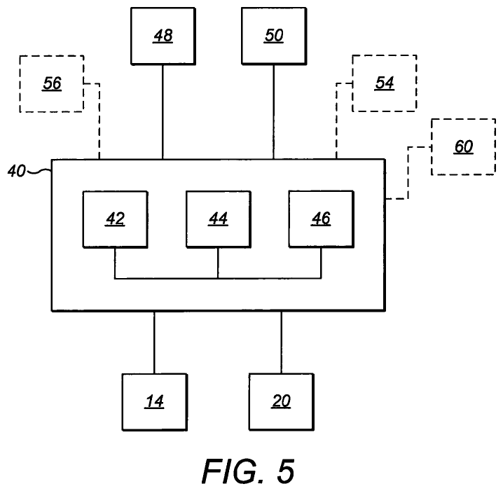

Referring to FIG. 5, the sterilising unit includes a control circuit 40, a power source 48 and an on/off control 50. The control circuit is operatively coupled to the power source, the on/off control, the cooling unit 14 and the LEDs 20, The control circuit includes a microcontroller having a processor unit 42, a memory unit 44 and a clock 46. The memory unit 44 stores computer program code configured to result in the functionality ascribed to the control circuit herein when run by the processor unit 42.

The power source is in the form of a battery, which may be rechargeable. Alternatively, the sterilisation unit may include a port by which the sterilising unit is connected for use to an external power supply, for example a mains supply.

The control circuit is configured to enable suitable supply of power to the LEDs 20 in response to a user operating the control 50 to turn on the UV light, such that the LEDs 20 emit UV light.

The control circuit is configured to prevent supply of the power to the LEDs 20 off when the user operates the control 50 to turn the UV light off.

In some embodiments, when the LEDs 20 turn on and off, the cooling unit 14 also turns on and off. Alternatively, the sterilising may include a thermostat 54 located to sense temperature in the armamentarium. The control circuit is configured to detect, based on a signal from the thermostat, when the temperature in the armamentarium exceeds a predetermined threshold temperature. When this occurs, the control circuit is configured to cause operation of the cooling unit 14 to reduce the temperature in the armamentarium. The melting point of endodontic cones stored in the armamentarium may be about 60° C. In this case, the microcontroller may be configured to prevent the temperature in the armamentarium exceeding a temperature at which there is risk of unwanted softening of the cones, for example 45° C.

While the thermostat is located to sense temperature in the armamentarium, coupling of the thermostat to the control circuit is such that bacteria cannot pass from the lower side of the transparent plate 12 to the upper side. Accordingly, a groove may be provided in one of the side walls 13 enabling location of a wire to which the thermostat is connected to fit tightly between the side wall and the transparent plate 12.

In variant embodiments, instead of the LEDs turning on and off in response to operation of a user control by the user, the sterilising unit may include a sensor 56 configured to detect when the lid 16 is opened. In this case, in response to receiving a signal from the sensor 56 indicating that the lid 16 is open, the control circuit may be configured to prevent supply of power to the LEDs 20. When the lid 16 is closed, the control circuit may then automatically cause the LEDs 20 to light.

In some embodiments, the memory unit 44 is configured to store a predetermined time period, for example 10 minutes. It is assumed that contents of the armamentarium on which UV light is incident are sterilised after the predetermined time period. When the LEDs 20 are turned on, the control circuit starts a timer. When the predetermined time period ends, the control circuit is configured to turn off the LEDs. In embodiments in which the sterilising unit includes the sensor configured to detect when the lid 16 is opened and closed, the timer is reset each time the lid 16 is closed.

In embodiments including such a timer, the cooling unit 14 may be switched on and off with the LEDs 20, as described above, or switched on and off in dependence on signals from the thermostat and on the predetermined threshold temperature. In variant embodiments, the cooling effect of the cooling unit 14 may be adjusted by varying the supply of power to the cooling unit 14.

The cooling unit 14 is not essential to all embodiments of the invention. Where articles located in the armamentarium are not sensitive to temperature, or where the heat generated by the LEDs 20 is not so high as to unduly raise the temperature in the armamentarium, the cooling unit 14 can be absent from the sterilising unit. The design of the container 10 can be modified accordingly.

In embodiments, the sterilising unit may include a display 60, operatively coupled to the control circuit. The control circuit may be configured to cause the display to indicate whether the contents of the armamentarium has been exposed to the UV light for sufficiently long so as to be sterile, or how much longer the contents should be exposed to UV light before they can be considered to be sterile. Also, the control circuit may be configured to record usage of the sterilising unit and may be configured to caused information indicative of the usage to be displayed on the display.

In embodiments, the sterilising unit may have an output port enabling information on the usage of the sterilising unit to be output to an operatively coupled computing device, where the appropriate information may be associated with patient's records. Instead of an output port, the information may be transmitted by Bluetooth or using another wireless communications technology. In this case, the sterilising unit may include a transmitter enabling output of the information.

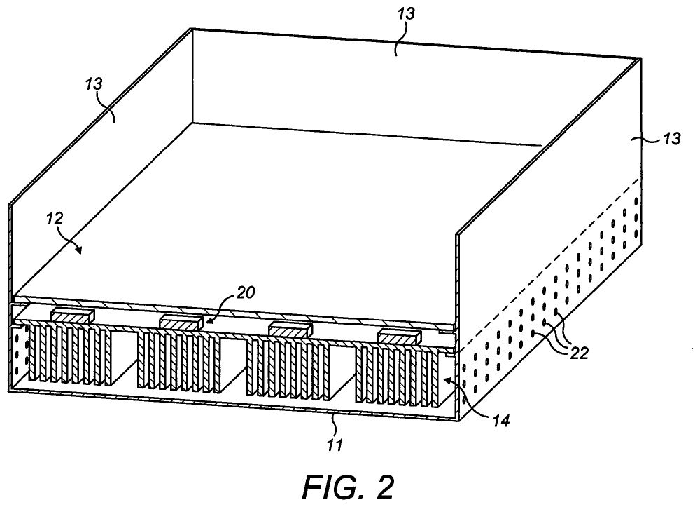

Referring to FIG. 2, the sterilising unit includes a plurality of removable receptacles in the form of first, second, third and fourth modules 26a-d which are, in use, located in the armamentarium and are supported by the transparent plate 12. There may be a greater or lesser number of modules. Each of the modules is made of a material transparent to UV light, for example fused quartz glass. Although, alternatively, articles and materials could be placed on the transparent plate 12, this may result in the plate 12 becoming dirty and marked. The modules 26a-d result in the plate 12 staying clean.

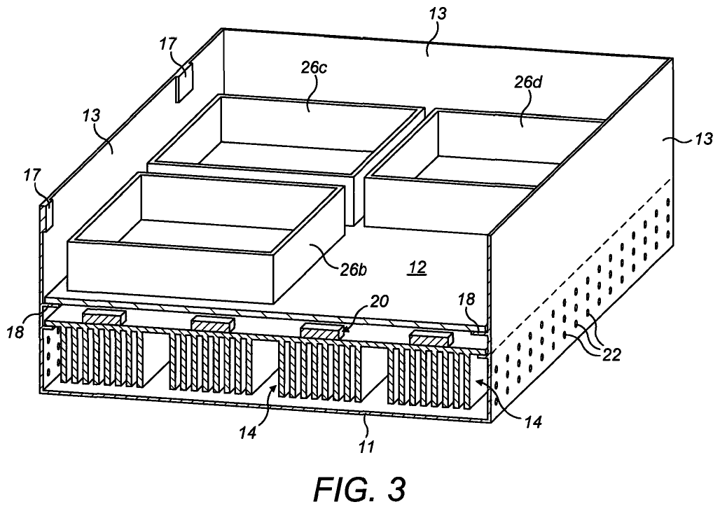

The first module 26a is shaped to provide a plurality of parallel grooves, indicated at 28. Each groove 28 is configured to retain an endodontic cone. Two such cones are indicated at 30 in FIG. 3. The grooves 28 are arranged to retain the cones such that the cones do not contact one another, to allow UV light to be incident all around the cones.

The second, third and fourth modules 26b,c,d are for storing other articles such as endodontic materials, instruments and paraphernalia used in endodontic treatment, for example mixing pads and spatulas for cement. The second, third and fourth modules 26a-d may be specifically shaped to retain particular endodontic instruments. In some embodiments, more than one module may be provided like the first module 26a.

The cones are used in root canal surgery, to fill and seal a root canal lined with sealer cements. The cones are available in standard sizes for use with different sizes of root canal reamers and files. The cones may be known as “gutta percha points”. The cones are often may of thermoplastic. The grooves 28 are arranged so that a plurality of cones can be laid out in the first module 26a.

The sterilising unit is for interoperative use, particularly use during root canal treatment. Before surgery a selection of cones is located in the grooves 28 in the first module 26a in the armamentarium. Instruments and materials to be used during the root canal treatment that may have been subject to microbial contamination are located in the second, third and fourth modules 26b-d. The lid 16 is then closed such that the armamentarium is sealed with respect to outside air. The control is then operated so that the LEDs 20 emit UV light, which passes through the transparent plate 12 and is incident on the cones 30. The effect of the incident UV light is to sterilise the cones, or at least to reduce microbial contamination. As mentioned above, as an alternative the control circuit may automatically cause the LEDs to emit UV light when a sensor is provided that signals that the lid 16 has been closed.

The sterilising unit is then left for at least a minimum time period so that the cones 30 and any instruments and materials in the armamentarium are sterilised by the effect of the UV light. As mentioned above, the sterilising unit may be configured so that the control circuit turns off the LEDs after a predetermined time period ends.

During root canal treatment, a user, for example a dental professional, opens the lid 16 of the sterilising unit and takes a suitable one of the cones 30. If the LEDs 20 are lit, preferably the user turns off the LEDs 20 before opening the sterilising unit, or, as mentioned above, the control circuit causes the LEDs to turn off when the lid 16 is opened.

The dentist then closes the lid 16 and presses the button to cause the LEDs to begin emitting UV light again, to kill microbial contamination that has entered the armamentarium whilst the lid 16 was open. In the variant embodiment in which closing of the lid 16 is sensed, the control circuit may cause the LEDs 20 to emit light automatically.

The selected cone 30 is then used in root canal treatment. The selected cone 30 has little or no microbial contamination on it when inserted into a root canal due to the treatment of the cone 30 with UV light.

The sterilising unit is described above as for use in endodontic treatment. However, the sterilising unit may usefully be used to sterilise instruments and materials in many interoperative scenarios. The first module 26a that is specifically configured to retain cones is not essential to all embodiments of the invention and may be absent where cones are not to be retained. The first module 26a may instead simply be configured to retain articles and materials. Also, instead of the sterilising unit comprising a plurality of modules, the sterilising unit may include only a single module configured to rest on top of the transparent plate.

The sterilising box is preferably portable. The sterilising unit can be used by a dental professional when chairside. The sterilising unit may usefully be used in hospital wards, or in-the-field scenarios where microbial contamination is conventionally difficult to avoid. The sterilising unit may also be usefully used to retain articles, materials and paraphernalia intended for use in procedures such as catheterization or venepuncture to reduce microbial contamination.

The applicant hereby discloses in isolation each individual feature or step described herein and any combination of two or more such features, to the extent that such features or steps or combinations of features and/or steps are capable of being carried out based on the present specification as a whole in the light of the common general knowledge of a person skilled in the art, irrespective of whether such features or steps or combinations of features and/or steps solve any problems disclosed herein, and without limitation to the scope of the claims.

1. Apparatus comprising:
a container;
an emitter configured to emit UV light located in the container;
at least one receptacle for location in the container and for retaining root canal filling material comprising a plurality of cones, such that each cone does not contact other of the cones, wherein the emitter and the at least one receptacle are configured such that UV light emitted by the emitter is incident on retained cones with anti-bacterial effect.

2. The apparatus of claim 1, wherein the apparatus further comprises a member transparent to UV light emitted from the emitting means extending across an interior of the container, wherein, in use, the emitter is located beneath the member and the at least one receptacle is located on top of the member.

3. The apparatus of claim 2, wherein the member and parts of at least one side wall that are, in use, above the member, provide a space in which the at least one receptacle and any root canal filling material located in the at least one receptacle is located.

4. The apparatus of claim 3, wherein the member extends across the interior of the container to seal the space in which the at least one receptacle is located with respect to a side of the member on which the emitter is located.

5. The apparatus of claim 2, wherein the container has a base and the emitter is located between the support member and the base.

6. (canceled)

7. The apparatus of claim 6, further comprising a closure to close the container, so as to prevent ingress of bacteria into the space.

8. The apparatus of claim 2, further comprising a support arranged to support the member.

9. The apparatus of claim 8, wherein the support comprises one or more projections extending inwardly from at least one side wall.

10. The apparatus of claim 2, further comprising a cooler located, in use of the apparatus, beneath the member, arranged to dissipate heat generated by the emitter.

11. The apparatus of claim 2, further comprising at least one air hole in at least one side of the container permitting air flow between outside of the container and the cooler.

12. The apparatus of claim 1, wherein the at least one receptacle is removable from the container.

13. The apparatus of claim 1, wherein the at least one receptacle is transparent to UV light emitted by the emitter.

14. The apparatus of claim 1, wherein the at least one receptacle is configured with a plurality of grooves, such that each groove has width to retain a cone disposed lengthwise therein.

15. The apparatus of claim 1, wherein the at least one receptacle comprises a plurality of receptacles.

16. (canceled)

17. The apparatus of preceding claim 1, further comprising a control operatively coupled to the emitter, and configured to control starting and stopping of emitting of UV light by the emitter.

18. The apparatus of claim 17, further comprising:
a member transparent to UV light emitted from the emitting means extending across an interior of the container, wherein, in use, the emitter is located beneath the member and the at least one receptacle is located on top of the member, wherein, when the base of the container is located on a horizontal surface, the member is horizontal;
a closure to close and open the container, so as to prevent ingress of bacteria into the space
operatively coupled to the control, a detector arranged to detect opening of the container, wherein the control is configured to control the emitter so as to stop emitting UV light in response to detecting opening of the container.

19. The apparatus of claim 18, wherein the detector is also configured to detect locating of the closure to close the container, wherein the control is configured to control the emitter so as to start emitting UV light in response to detecting closing of the container.

20. (canceled)

21. (canceled)

22. The apparatus of claim 17, further comprising:
a member transparent to UV light emitted from the emitting means extending across an interior of the container, wherein, in use, the emitter is located beneath the member and the at least one receptacle is located on top of the member, wherein, when the base of the container is located on a horizontal surface, the member is horizontal;
a closure to open and close the container, so as to prevent ingress of bacteria into the space, wherein the control is operatively coupled to the cooler, and is configured to cause starting and stopping of the cooler with starting and stopping of emitting of UV light by the emitter.

23. The apparatus of claim 22, further comprising a thermostat operatively coupled to the control and located to determine information indicative of the temperature of the at least one receptacle, wherein the control is configured to cause starting and stopping of the cooling means dependent on the information received from the thermostat.

24. Apparatus comprising:
a container;
means for emitting UV light located in the container;
at least one receptacle for location in the container and for retaining root canal filling material comprising a plurality of cones, such that each cone does not contact other of the cones, wherein the emitting means and the at least one receptacle are configured such that UV light emitted by the emitting means is incident on retained cones with anti-bacterial effect.
